SEQUENCE LISTING (4 sequences)

SEQ ID NO: 1 - DNA, 4900 bp, Homo sapiens
gactcactatagggagaccggcagatctgatatcatcgccactgtggatccgaattctagaaggcctatgttctaagcatcaggctttacctgtgaatctcctctttttacagatgaagatgactgtatcactcagattcccggcaggaaagcaatggcatactcaagtggggtaactaatgatggaaccatttacaaaggtgtggacagagttaagaaaaagcaataggagatagtgagcttcctggggctggtaagagtggggagcccttaccactcccaggactaaaggagggagtggtgcccagaagccctgcctatatgcaactgagaagggcagggccagggagtcacgtccatcctcactgctctccagtctcctgaactggaagccagaaggtgaggggaaccctgatgcagtttgtatgtgtgagaaagtacaattagtttagactgaaaaactgaaaatctacccggccacttagcaggctggaataacagaaatggatcaagccagctgtaaagataacagggaacaataattctctgtagctgtaaagtgataatacaaccctgcatctttgagtgactgctgaaacattgtcctttaaaatcagagaccttcagaaacttcgctgtttgaaattacatgactaagactgaaatattccaattttgcctggaagatttaagtcatcttgacacagagaagcagcctcaatttacaactcaggagcagagcttcagataaagattttctggacacatttgacatgtatcttagctatgttgcttcctaggaaacagggccctgggtcctctttgcaatccagactgaagttgactgctttgtacaaacctgttttgctttgagtccatcaaaacatgacttcatttagattttatctcaactccactttcctcggaatcctatamtaaattgctgttttcctttgtttggtgatgtgcgtagctcttctggtgggtggtgtccctcactgaataggtcaayaaacctaactttgttggactgccactgtgtccctggtgatctttggctgattggtctaggtcatagatcgacctgccggggtgcagaggagggtggagagtaactcagagggtcaagcatgaaagatctggcagraaaataaagcccctccacccccaccacccctacccctgcaaatctgatttcccccaccaactgcagaccagagtattataaggggcggtggaagaagagggggagatcttcatttacccagagctcctatacatcaggggctgaataaagggttgtagaaatgaatgaatcaatctctgagtggggcttcaggcartggaaagatctcagtccttttctgaggcataatggaagctcccagtcttgtgacatttgcaaggctgcccctttctcccaagagacatgagaccaaaaaagtgaaaggaaaggggggaaaagggagaattctraaaatgcccatcctctgaacaccatctttgtgtaggcatctgggggaggccagctggggtgaggtcatctgccagccaggcccgtaggacttggcgcttcttgtttatcacagccacatgtggggccactgccagggcccgccccaactctgcagtcattggaggagcttgaagttaaagactcctgctaaaaaccagtacgtttcattttgcagttactgggagggggcttgctgtggccctgtcaggaagagtagagctctggtccagctccgcgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttggtaagtgggatctggggaggacccagatctctgcagtggccgacagcacagaaagccccagcgggcagcttcaggtgcacattctgaatctcacatggttttcgaatctgagacgtgctctcacagccagctgggcgggagggaggaagcagcagcaggcaagaggaaacggtgccaggctgcagcagagagaagccacaggacaagcgggattcctttctgctctacttcaggcccgccagggcgcgcaaggcagggcgtgcctggggaaggtaggaaagcgcagggcaacaccctggatccccagggaggaggcgaggatctcagggcacgcctggtgatcatgctggcatctgagtcaccatgcttgggaggaataggaccaggcttgaaaatgtgttataactttaggtcctcaccaacgtcaggaaggccctgctttttggtttttgtttcttctaaaagaaacttactgagatataatttatacaccatacaattgacccatttaaagggtaccatttaatgattttcagattattcccagagttgtgcaaccatccccacaatcaattttagaatattttaatcgactcaaaaggaatcccacactccttcaccatcatttccaacactgttcctcctccttcccacccatcaatttcctttctgcctctatggatttgccgattctggacatttcatataaatggaatcacataatatgtggtcctttgtggcacttagcgtgttttcaatgctcatccatgttgtagcatgtgttgatacttcattcaatttttttttttaaagagacggggtctcactattttgtccaggctggtctcaaactcctggactcaagtgatctgcctgcctcggcctcccaaagtgtcaggattacaggcgtgagccattgcacccggctgatacttcattcctttttacggctgagtagtactccattgcatggatagaccacttttttctatccattcatccattgatggacattggggttgtttctcttttttggctatcatgaataatgccacatgaacatttgtgtacaaggttttatgtggatatatattctcctttctctcgaatatgtacctaagagtaaaaattgctaggtcatatgttaactatgtttcacctttggggggaatgtggagctgaatttcacagcagctgcagttttttacattcctatcagcagagtatgagggatccaatttctccacatcctcaccaacgcttgttatcgtctgtctttttgggttttgttgttgtcattttgtttttgtctttgagatgaagtcttgctctgttgcccaggctggagtgcagtggcgcaatcttggctcactgcaacctccacctccccggttcaagcgattctcctgcctcagcctcatgagtagctgggattacaggtgtgcgtcaccactcctcactaatttttgtatttttagtagagatgaggtttcgccatgtaggccaggctggtctcaaactcctgacctcaagtgatccgcccaccttggcctcccaaagtgctgggattacaggcatgagccgccacgcccggctgattgtctgtcttttttattatagccatgctagtgggtgtgaagtggtagttcattgtggttgtgatttgaatttccctgatggtgagtgcctcttattctctgtgctgattgataatgatgatgaaggcaatttgtatctatagagtggcagtgtagtttactaagagttagggtaacttatttcatagtactggctatgtcttctgggccaagtcattaacttctctgagcctcagtttctgcatctgttcatagggttgtggcaattaaccaaaaaaaaaaggcatgaacagcccttatcatgatgactgacataggataagagctccataactagtatctatttttaaaaataatctttttaagtctgggagtggtggctcacacctgtaatcccaacactttgggaggccgaggcgggtggatcacgaagtcaggagtttgagaccagcctggccaatatggtgaaaccccatctctactaaaaatacaaaaattagtggggagtggtggtgcacacctgtaatcccagctactagggaggctgaggcaggagaatcgcttgaacccggaggcggaggttgcagtgagccgagatcaagccactgcactccagcctgggtgacagagcaagactccatctcaaaataataataatagtaataatttttttgattatataatagtatatatgtatataaaatacatgtatgtatttttatctatatcctctgctctgaccctcaaagtaaccacgtccaagttcaggatttgaaatctggaaacgtggattcaaaaatccttcacctctttgagccttggtttcatcatctgtaaaatggggagaattgttgataggaatattaaatgaactaataaatgcaaagctgtttgagaaatatatggcatatagtaatccctgattaagtgttagttcttattattaataatgctattattaggattattattattcgattcatatgtttactgttcaacaaatattgaatgataaacatatatgctgggtccggcatggtggcccatgcctgtaattccagcactttgggaggccaaggcgggcaggtcacttgaggtcaagagtttgagaccagcctggccaatgtggtggaaactccatctgtgctaaaaatacaaaaattagccgggcatggtggtgggtgcctgtaatcccagctactcgggaggctgagacaggagaatcacttgaacccaggaggtggaggttgcagtgagccaagattgcaccactgcactccagcctgagccacagagcaagactctgtctcaaaaaaaaaaaaaaaaaatatatatatatatatatatatatatagtatttttagtagagatggggttttgccatctcttatatatttttatatt

SEQ ID NO: 2 - DNA, 1853 bp, Homo sapiens
aaaacgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgyggagaatggagtgaagaagttggtgcacagtgtctttgacaccgcagactacaccttccctttgcaggggaactctttcttcgtgatgacaaactttctcaaaacagaaggccaagagcagcggttgtgtcccgagtatcccacccgcaggacgctctgttcctctgaccgaggttgtaaaaagggatggatggacccgcagagcaaaggaattcagaccggaaggtgtgtagtryatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgkggcaattcagggcggaataatgggcattgagatctactgggactgcaacctagaccgttggttccatcactgccrtcccaaatacagtttccrtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggccrctgtgttcatcgacttcctcatcgacasttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtccsaagacctgygatggacttcacagatttgtccaggctgcccctggccctccatgacacacccccgattcctggacaaccagaggagatacrgctgcttagaaaggaggcgactcctagatccagggatagcccygtctggtgccagtgtggaagctgcctcccatctcaactccctgagrgccacaggtgcctggaggmgctgtgctgccggaaaaagccgggggcctgcatcaccacctcagagctgttcaggaagctggtcctgtccagacacgtcctgcagttcctcctgctctaccaggagcccttgctkgcgctggatgtggattccaccaacagccggctgcggcactgtgcctacaggtgctacgccacctggcgcttcggctcccaggacatggctgactttgccatcctgcccagctgctgccgctggaggatccggaaagagtttccraagagtgaagggcagtacagtggcttcaagagtccttactgaagccaggcaccgtggctcacgtctgtaatcccacctttt

SEQ ID NO: 3 - DNA, 11270 bp, Homo sapiens
  modified_base (5554)..(5569): This range may encompass 3 or 4 gttt repeats
catcacctacaaaggaaaccccaaatccagtagcagtcactccccattctccccttcccctgtccctggccacagtctactttctgtctctatagatgcctattctggacatttcctataaatagaattgtatatggtgtggccttttgtgtctgtcttctttcactcagcatcatgttctccaggtccatccatgttgtagcctgtgtcattgcttcatccttcttatggctaaataagattctgtgtatgaatgtaccacattttatttgtccattcatccgtcagtggccacttgcatggtttccacttttttggcgattctgagtagtgctgctataagcattcgtgtgcacattctggtggatatcgaatcacttctccacatcttagtaacacacgtcacttactccccactctgtcatccttctatctgcagtatcccacccgcaggacgctctgttcctctgaccgaggttgtaaaaagggatggatggacccgcagagcaaaggtaccttctgtttcttttcccgagaccctaggggtggatggtctggcatcttggtgacatttgtgatgcccaggtcaggtcttcagcctctgctctcagctgccctcttccaccatcaccaagccataggcgagtctgcccatgcttcggctctgtccccagcagaccagctgctgactgtaaacatgactccagttttccagtgagagaagaagctcctaaaaacctagcaggttcaggattctaatcggtagaaaattcacatggcctatagcatcatctgagtattctaaactttccccctgaatttcctcaaaggttgaggaccatgaacttttacccccaggggaacctggcagcaatacccatattaacctgcagaattttttttgttttttattttattttattttttaaacattttttgcactgttttattttgattttgattttgattttatttatatctaagtgcagtgctattgcgatactgcagaatttctttatctcacattttaacttaaaaaggcacagggcagcgagcgcagaggctggtgcctgtaatcccagcactttgggagggtgaggcagatggatgcttgaggtcaggggttcgagaacagcctggaaaacatggtgaaaccccgtctctactaaaaatacaaaaatcagccagacatggtggcacacgcttataatcccagctacttgggaggctgagacgtgagaatcacttgaacctggaaggcagaggttgcagtgagccaagatcatgccactgcactccagcatgggtgacagagcgagacccctttaaaaaaaaaaaaaaaaggcacagggcaattttaaaaatactgcaaatagtaaaaaaaaaaaaatcagtggttataatgcaaacacacacaaaaaggcatatgcccattactgcattctactccatactgtatgtgtatttgagttagtataaaagttattttaacattgctcactatttaattaattctcccttggaaactgattaatcatcctggcactccaggaagatgtgccatgctgatttcatggctttgcacatcctgggcaggctgtgtaccccttgagggacttgtgcccctttgagaggccatgttctagtccatttatactaagtgagagcatacacctgttccgctcccctcatgggcaccttttcttataaagaaacaaaagagccagcagaatccacagtctttctgtgttctctctgatctttattatgttttgcttgtttgccttgccttgtgttcgttgtggttaggatgggcttgatggaagctgaagctgcgtgggttggaaagcctggtcaaagcctagtctctcgcccgggttgagttaatgatgtccctcctggagaacgtcctctctgcagttctttcacatctgtggttctacgatgctttgacccctataggaattcagaccggaaggtgtgtagtgcatgaagggaaccagaagacctgtgaagtctctgcctggtgccccatcgaggcagtggaagaggccccccggtgagtcgcatggggagacagacacagtggccctcagcggcgaccagatgaggccttgccgaggctgcttgggccttcccctctcagcacagccctgcaaagtcctgggtcctaccggcttggggacccctgcgctctggatgcactgcttggcacaaactagtatctctgggagggccatggtggttggtaaactgttgtaacactcctgtaccaactggtaaatagctactaccctgagcatccttgggtgtccctggccccttccttcccccagatcttccagggtacccccagaccccctcctgtagtgccacagcaggatcccttctgacttgtcagtgtccatactgagtgatcaaggataggaaggaaggagggagatggaaaggaaggacgaagcgaggaaagagaaggggaaggggaggaaaaagcaaaaggggtgagggtaaaagagggggggaaaggaagttttctcaaattaaatgcttacaatgacatacagatttggtggtcccttgtattgatgcttcgcttcaatacacaaagtcacaatgttaaatctcagaagccacaagggctgatgtatttcagcagagaatagttagaaagacctggattcaattcctagctctaacaccattttgctgtgtgtccttgggaaaatggcttaacctctctgagtttcagtgtcctcacctgtaaaagcagaataataatttcaccaacttcatagggctgttgtaaggattaaatgagatgatacttgtacagttattgtaaggtaagccccatgcatgcctggcttacacacacacacacacacacacacacacacacgcacacacacacacacacacaatctacccctagaagtgtggtggttctagaccagcactgtccaattgaacttgatgcagtgatggaaatttctgtatctgtgctgtccaatagggcagctactaggtacatgtggctattgagtacatgaaatgcgactactgaatttttgaaagagatgatagatgatagatagaaagatagatagatagataaatagatagataatagatagatagacaggtagatagatagatagatagatagatagatagatagatagatagagttttgctatgttgcccaggctggttttgaactcctgggctcaagcgatcctcctgccttggcctcccaaagtgctggggttacaggtttgagccattgctcccagcctgaatttttaattaaatttaaatttaaatagccacacatgtctagtggctaccatattggacagcgcagttctagaccgatgtgattcaggatcattccctcagcatcgtggggcaaagagaaaactgccccaagctggcctgtagaaggctcaggcgaaggtttcccaatgccgggatggggggtgcgctcagcagcatcaccccttatgattctcaatcgctaatagctccactcaggttcatttctcggtcaggggcatttctttgggaatcacccagctctgggagatacagcagcctccactcaggtagtccttgttcaagacaagcggcccttgactgactgcagtttcagttccagctctgctatcaactcactcattaaataaactgcatctccagtgtgcctgcctctgggctggattttgacgtgacctgggcaagcaactccctgaacttcagtttctcatatattatatgaattagctaagatggttcgtttaatcattcattcaacacatccatcaccacgtagtaggtgttagatatttatttcatacgtaactacgcataagagactttgctaagttttaggtaaaatacaagtcccagatacggagcaagtctcaaccactgtacatacctgaatgtgtaattacatcactgtaagaggtgccacagtaaatgccactgggtcttgtgttagtccattctcacacaaagaactacctagccaggtgcggtggctcacgcctgtaatcccaacactttggcaggctgaggcaggcggatcacttgaggtcaggagtttgataccagcctggcaaacatggtgaaaccccatctctattaaaaaatgcataaattagccaggtgtggtggcacacgcctgtaatcctggctactcgggaggctgaggcaggagagtcgcttgaacccgggagatggaggttgcagtgacccgagatcgcgccactgcactccagcctgggtgacagagtgagactccatctcagaaaaaaataaaaataaaaataaagaactacctgagaccaaacactttacgaaaaaaaagaggtttaattgactcacagctccacaggcttaacaggaagcctcaggagacttacaatcatggcagaaggcgaaggggaagcaaacacatcttaccatgatggagcaggaggcgggtttcgggggatgtgccgcacacttttaaatgatcagatctcgtgagaactcactcactatcacgagaacagcaaggaggaagtccgcccccatgattcagtcacctcccaccaggcccctcctctggcacatggggattacaattcaagatgagatttgggtggggacacagagccaaaccatatcagatctcaagaagggagaaattcttcttggaggagctggaggggctttgtggagagtttcagaatgctttgcccactaggtttgctgtatccatttctcttcatgyatcccaaagaccaagccaagaaaccagaagcctctggtcccactggcccatgggctccctcggtycccmccgtcactaatggccattttgcatgtctctctcccaggcctgctctcttgaacagtgccgaaaacttcactgtgctcatcaagaacaatatcgacttccccggccacaactacaccacgtaagtgcccaggctgcctggctgtcttagttatctactgctgagtaataaatyatcccaaacctcagaagcctgaaacaacaaacgcctattgtctcccacggtttctgtgggtcaggaatctgggaatgactttgctgcgtggttctggctcaaggtctgtcaggttgtagccaagctgtcaaccagggctgcagtcatttctaggcttgactggggctggagaacccttttccaagctctcacacagttgctcgtgggagagctcagttcctcaccacgtgaacctcgccctagaccacttgagtatccttggtatatggtggctggcttctcccagagcaagtgacccaagagagacagagcaagcaaccaagagtataaccaagatggaagccacagtctttggggggagaccccaacacttctgccatatgccattggtcacacagatcaaccctggtccagtgtgagaggccactgcccaggggtcccaggaggcagggatcatttggggctttcatggaacctctccaccacactggctcactcctgggaaagagacagwtctgttttcaatcgagatgtttgtttgtttgtttgcttttaattatgsacaggagaaacatcctgccaggtttaaacatcacttgtaccttccacaagactcagaatccacagtgtcccattttccgactaggagacatcttccgagaaacaggcgataatttttcagatgtggcaattcaggttggtggtgctttgtacactgggatgtggggctgtgtgtctagggatggaggatgtcaaacagccaagaggccgggccactgggtcttcataatgtggctcacatttactgagcatttagtaaatccaccygctgcgctaaggtctttacctacgctacctcgtcaaatcccaaaacaatccttatgagtgagagctacttggtgtattcctttcctgtggctgctgtagcaagttatcaaagcttagtggcttcaaacaacacatatttgcttatgttgccagagatcagaagttggagatgattttccctgagccagggcggtgctccctctgggactttaagggagaatccagttcctcagcttttccaccttctggagctgcattccttgcatttcttcaaagccagcagcataacatcttgcctcagtggccactttcactccctatcctgtgtccaatctccctttgcctctgtcttaaaaagagagagagcatttacaagagggggcatttaaggaccaactggataatccaggataatctcccatctcaagatccttcatttaggctgggcacggtggctcatgcctgtaatcccagcactttgggaggctgaggtgggtggatcacctgaggtcaggagttcaacaccagcctggccaacatggtgaaagcccatctttactaaaaatacaaaaaaaaaaaaaaaatagccgggcatgattgcgggctcctgtaatcccagctactcgggaggctgagacaggagaatcgcttgaacctgggaggcagaggttgcagtgagccgagatcgcaccactgcactccagcctaggtgacaagagcgaaactccatctcaaaaaaaaaaaaaaaatccttcatgtattcgcatctgcaaagagctttccctaggggagtactaggaggtaaagcagaaaagatatttgatagagtgccctgaattccagtctaataagtttggacttgatctttaatgggggcgtggggggcattaaaggtgtttgggtacaggagtggtctgttgaaagttgtattttaggacaatgagtttaacagtgatgtgtcccagacgggggtagggagagtgaggagatgcgattgtggctgccacaataacacttgtgcgagttaggtggggctgtacatatggttcttcaatcagcatttttyctctaaaaaccttaagcaatcctggctatgcagggagatgtctggcggttgcgtaactcacacccagcagccatagagactgtcccttgttgatccttcagggcggaataatgggcattgagatctactgggactgcaacctagaccgttggttccatcactgccgtcccaaatacagtttccgtcgccttgacgacaagaccaccaacgtgtccttgtaccctggctacaacttcaggtaactccaaggcccaggtcaaactcacccagtggctgaatcgcattcccaggaactggtgagactaattttggtttccaaggcaacaagatgaatgaaaaaagactttctctaagaactaggtgataactgaattttttccataattttttaaaattctcaaaagagatacactctttattttttacttatttttttttttttgaaatggagtctcactctgtcacccaggctgaagtgcagtggcgccatctcagtcactgcaaacttccgcctcccaggttcaagcgactctcctgcctcagcctcccaagtagctgagattacaggcggatgcacactgtttataaaacaaaactattgggaaacagaaaagcatagagggggatcaaaatcgcccataattcccctaccctgaaataatcaataacaaccctcgggggaattttcctcatctgtaccaattatttcatacagctcctatgagatcatagcatatatatatatatatcttgtggtattctgcagggtttttcataccacagccactcaaaattctttgtaaccatcacattaatgatcataacattccattttgtaggtgaacaaataacaactgctacaattcaggaagtgttttcttttcttttcttttcttttcttttttttttttagatggagtcacactctgcttgcccaggctggagtgcagtggcatgatctcagctcactgcaacctctgcctcctaggtccaagcgatcctcccacctcccaagtttctgggaccacaggcatgtgccaccacacccagctaatttttgtatattcagtagagatggggtttcactgtgttggccagtctggtctcgaactcttgacctcaagtgatcttcccaccttggcttcccaaagtgctaggattacagtcatgagccactgtgcctggcccaaggagggttttccatataccaagcactccccatcgccatccctaaatctcccaacaaccctggaaggaagatattgtttctggaagatgatttgcccaagacccacagctgatagtacatgtttgcataattctaacccacgttcactctgaccccacactcacactcccatcccttcccttcccatctcaatgattttctcaccgtacgcctccatgaattgaatatttgagttgcttcccagtttttctagtacaagtaaccacagtgtgcatctttgcaccgtaaacttcttctttgaattccagggttacttccctaggataaatttcctagacttattgaatcaaaggttgtgaacattttatcatatgctttttatttttaaaaatatctatggttataatgtttcatttttttttctgagacagagtctcactctgtcacccaggttggagtggaccgggtgcaattatagctcactgcaacctctgcctcccaggcccaagtgatcctcccacctcagcctcctgagtagctaggactacaggtgcatgccaccatgcccagctaatttttaaaatttttttgtagagtcggggtctcactatattgcccaggctggtctcaaactcctggctcaagcaatccgcctgccttggcctcctaaagcgttgggattgcaggtgtgagctactgcacctggcctataattttcatttctaggatttttatttggtgctttttcaaagtcatctattattgttccagtgagtcccattcttaccttaaggatcctactccttctgtccattctactgtatcattcctttcataccgacctattatctgaagtaacttgggtgggagttctcctcgtgggctttgaaatactgtcttcagtagaaaagatcttatgcaaagttctgcatgtgctgtgtgttatggagtattccttatatattactcaaagcagcttcatgaactggcaggcagccccttaaatgtgtttctgtttctgtttttttgttttttggtctgcatgatgttttaaaacttgagacaggccaggtgcggtggcttatgcctgtaatcccagcactttgggaggccaaggtgggaggatcacttgaacccgggggtttgaggctagcctgggcaacatagggagaccccatctctacaaataataatttttaaaacatcagccaggtgtgggggcatgcacctctggtcccagctactcaggaggctgaggcaggaggattgcttgaacctgggaggttgaggctgcagtgaaccgtggttgtgccactgcactccagcctgggtgacagagtgagacccttacatggtggccactggctggagctgagtatcagtggtcctatttagaaaggggctgggctttctggttcatcactgtccccgccgctccttagtgcttatacctggcccacatcactcatttctgtcatctgcctggcccctgtgtagacatttgagtttgaaacccttgactcaaaggcaggctgatgctttttgcttcctctggatcaatggaatttcatccaaggcaatggaaccagctcctaattgtgataactttttgatcatcccctgccactgtgctgaataggcttatggccattaagaagagaggacaggatgaatggtcccctgactgactgcccttccagggtgtttttttagctgtcagcagaagcatgcggggcagtgtaccaatcaggtgtcagcaagtgtccttccagcgactgagttgagcacaaaaacatctgctcctggagagacctgagccctctgaaggcctcagccagttatgatgttaatgttcttttagaacaaagtggtacacccatttgttgtctggaatgagcaccagaacaaatctttggccaaaaataaattgttgagggccgggagcagtggcttatgcctgtaatcccaacactttgggaggccgaagcaggaggatcacctgaggtcaggagttcgagaccagcctggccaacatggtgaaaccctgtctctcctaaaaatacaaaaattagctggacgtggtgacaggtgcctgtaatacagctactgggaggctgaggcagggagaactgcttgaacccggaaggtggaggttgcagtgagccggggtcatgccacttcactccagcctgggcaacagagcaaagctctgtctttaaaaacaacttaaataaataaataaattgttgaggtctgatgagtaagtggacaagttattttccagcagacacacaaaagagaaggaaattacaggttataggaggtatttcagaaaatataactttctaaaacataggaagttgaagaagttgatcacattacagaattctgttgtttagaaaatgacctgtggcgaaatgtccttattcagtgaataggtgattccgcttatgcacgacctgtgtgaagtggatcaggccacccagaatgcacgatgcgcttctcaggcccagcaggagtatgtgtctgtgttaatttcctgtggctattatgactaattgccacaaatgtggtggcttaaaacaacagaaattaatcttcttatagttctggaagccagaagtttggaatcaagatgtcagcagggccacactcgctctgatgctctacgggagggtcctctcttgcctcttccagcgtctggtggctccaggcattccataactttatagcagcgtcccacaaatctctgcctccatcctcacatggccttctccactgtgtctctatgtcttcaatctcttttttttttttttttttttgaggcagggtttcactccagtctcctagactgaagtgcaatggcgtaatttcgggtcactgcaacctctgcctcccgggctcaagcgatttgatctctcctttatcttataaagatactagtcattggatttggggcttaccctaaatccaggataatctcatcttgagatgtttaacttaattatatctgcaaacactgtatttccaaataaggtcatatcacagccactagggattagatacttgaacatatcttatttgggggctcaacccattccagtgtacgaaaaacactcttgttcaaggcccgatgtttctcagggcatagcccactgactacctg

SEQ ID NO: 4 - protein, 595 aa, Homo sapiens
  MOD_RES (76): Ala or Val
MPACCSCSDVFQYETNKVTRIQSMNYGTIKWFFHVIIFSYVCFALVSDKLYQRKEPVISSVHTKVKGIAEVKEEIXENGVKKLVHSVFDTADYTFPLQGNSFFVMTNFLKTEGQEQRLCPEYPTRRTLCSSDRGCKKGWMDPQSKGIQTGRCVVXEGNQKTCEVSAWCPIEAVEEAPRPALLNSAENFTVLIKNNIDFPGHNYTTRNILPGLNITCTFHKTQNPQCPIFRLGDIFRETGDNFSDXAIQGGIMGIEIYWDCNLDRWFHHCXPKYSFXRLDDKTTNVSLYPGYNFRYAKYYKENNVEKRTLIKVFGIRFDILVFGTGGKFDIIQLVVYIGSTLSYFGLAXVFIDFLIDXYSSNCCRSHIYPWCKCCQPCVVNEYYYRKKCESIVEPKPTLKYVSFVDESHIRMVNQQLLGRSLQDVKGQEVXRPXMDFTDLSRLPLALHDTPPIPGQPEEIXLLRKEATPRSRDSPVWCQCGSCLPSQLPEXHRCLEXLCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

(19) United States
(12) Patent Application Publication
(10) Pub. No.: US 2003/0008293 A1
(43) Pub. Date: Jan. 9, 2003

(54) Chemical compounds

(76) Inventor: John Edward Norris Morten, Macclesfield (GB)

Correspondence Address: PILLSBURY WINTHROP, LLP, P.O. BOX 10500, MCLEAN, VA, 22102, US

(21) Appl. No.: 09/977,221

(22) Filed: Oct. 16, 2001

Related U.S. Application Data
(60) Provisional application No. 60/244,897, filed on Nov. 2, 2000.

(30) Foreign Application Priority Data
Oct. 21, 2000 (GB) 0025859.0
Apr. 6, 2001 (GB) 0108654.5

(51) Int. Cl. C12Q 1/68; C07H 21/04

(52) U.S. Cl. 435/6; 536/23.2; 536/24.3

(57) ABSTRACT

This invention relates to polymorphisms in the human P2X₇ gene and corresponding novel allelic polypeptides encoded thereby. The invention also relates to methods and materials for analyzing allelic variation in the P2X₇ gene, and to the use of P2X₇ polymorphism in treatment of diseases with P2X₇ drugs.

This invention relates to polymorphisms in the human P2X₇ gene and corresponding novel allelic polypeptides encoded thereby. The invention also relates to methods and materials for analysing allelic variation in the P2X₇ gene, and to the use of P2X₇ polymorphism in treatment of diseases with P2X₇ drugs.

The P2X₇ receptor (previously known as P2Z receptor), which is a ligand-gated ion channel, is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and L-selectin shedding (lymphocytes). P2X₇ receptors are also located on antigen-presenting cells (APC), keratinocytes, salivary acinar cells (parotid cells) and hepatocytes. Compounds acting at the P2X₇ receptor are therefore indicated as pharmaceuticals for use in the treatment of rheumatoid arthritis, osteoarthritis, psoriasis, allergic dermatitis, asthma, chronic obstructive pulmonary disease (COPD), hyperresponsiveness of the airway, septic shock, glomerulonephritis, irritable bowel disease, Crohn's disease, ulcerative colitis, atherosclerosis, growth and metastases of malignant cells, myoblastic leukaemia, diabetes, Alzheimer's disease, meningitis, osteoporosis, burn injury, ischaemic heart disease, stroke and varicose veins. For further background, the reader is referred to the following articles: North and Barnard in Current Opinion in Neurobiology 1997, 7, 346-357; Rassendren, JBC, 1997, 273, 5482-6; and Buell, Receptors and Channels, 1998, 5, 347-354. The terms P2X₇, P2X₇ receptor and P2RX7 are used interchangeably herein.

All positions herein of polymorphisms in the 5′ UTR region of the P2X₇ polynucleotide relate to the position in SEQ ID NO 1 unless stated otherwise or apparent from the context.

All positions herein of polymorphisms in the exon regions of the P2X₇ polynucleotide relate to the position in SEQ ID NO 2 unless stated otherwise or apparent from the context.

All positions herein of polymorphisms in the intron regions of the P2X₇ polynucleotide relate to the position in SEQ ID NO 3 unless stated otherwise or apparent from the context.

All positions herein of polymorphisms in the P2X₇ polypeptide relate to the position in SEQ ID NO 4 unless stated otherwise or apparent from the context.

All positions herein of polymorphisms in the P2X₇ polypeptide relate to the position in SEQ ID NO 4 unless stated otherwise or apparent from the context.

One approach is to use knowledge of polymorphisms to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The present invention is based on the discovery of polymorphisms in P2X₇. In particular, we have found thirty polymorphisms in the coding sequence of the P2X₇ gene, 12 of which lead to changes in the sequence of expressed protein.

According to one aspect of the present invention there is provided a method for the diagnosis of a polymorphism in P2X₇ in a human, which method comprises determining the sequence of the human at at least one polymorphic position and determining the status of the human by reference to polymorphism in P2X₇. Preferred polymorphic positions are one or more of the following positions:

positions 936, 1012, 1147, 1343 and 1476 in the 5′ UTR region of the P2X₇ gene as defined by the position in SEQ ID NO: 1;

positions 253, 488, 489, 760, 835, 853, 1068, 1096, 1315, 1324, 1405, 1448, 1494, 1513, 1628 and 1772 in the coding region of the P2X₇ gene as defined by the position in SEQ ID NO: 2; and

positions 4780, 4845, 4849, 5021, 5554, 5579, 5535, 5845 and 6911 in the intron region of the P2X₇ gene as defined by the position in SEQ ID NO: 3;

positions 76, 155, 245, 270, 276, 348, 357, 430, 433, 460, 490 and 496 in the P2X7 polypeptide as defined by the position in SEQ ID NO: 4.

The term human includes both a human having or suspected of having a P2X₇ mediated disease and an asymptomatic human who may be tested for predisposition or susceptibility to such disease. At each position the human may be homozygous for an allele or the human may be a heterozygote.

The term “status” refers to the genetic status of the human as detected by potential sequence variation at defined positions of a polynucleotide or corresponding protein. The term “diagnosis of a polymorphism” refers to determination of the genetic status of an individual at a polymorphic position (in which the indiviual may be homozygous or heterozygous at each position).

The term polymorphism includes single nucleotide substitution, nucleotide insertion and nucleotide deletion which in the case of insertion and deletion includes insertion or deletion of one or more nucleotides at a position of a gene and corresponding alterations in expressed protein.

In one embodiment of the invention preferably the method for diagnosis described herein is one in which the polymorphism in the in the 5′UTR region of the P2X₇ gene as defined by the position in SEQ ID NO: 1 is any one of the following:

at position 936 is presence of C and/or A; at position 1012 is presence of T and/or C;

at position 1147 is presence of A and/or G; at position 1343 is presence of G and/or A; and

at position 1476 is presence of A and/or G.

In one embodiment of the invention preferably the method for diagnosis described herein is one in which the polymorphism in the coding region of the P2X₇ gene as defined by the position in SEQ ID NO: 2 is any one of the following:

at position 253 is presence of T and/or C; at position 488 is presence of G and/or A;

at position 489 is presence of C and/or T; at position 760 is presence of T and/or G;

at position 835 is presence of G and/or A; at position 853 is presence of G and/or A;

at position 1068 is presence of G and/or A; at position 1096 is presence of C and/or G;

at position 1315 is presence of C and/or G; at position 1324 is presence of C and/or T;

at position 1405 is presence of A and/or G; at position 1448 is presence of C and/or T;

at position 1494 is presence of A and/or G; at position 1513 is presence of A and/or C;

at position 1628 is presence of G and/or T; and at position 1772 is presence of G and/or A.

In one embodiment of the invention preferably the method for diagnosis described herein is one in which the polymorphism in the intron region of the P2X₇ gene as defined by the position in SEQ ID NO: 3. is any one of the following:

at position 4780 is presence of C and/or T; at position 4845 is presence of C and/or T;

at position 4849 is presence of A and/or C; at position 5021 is presence of T and/or C;

at position 5554 is presence of 3 and/or 4 repeats of GTTT (wherein position 5554 refers to the position of the G in the first unit repeat);

at position 5579 is presence of G and/or C; at position 5535 is presence of A and/or T;

at position 5845 is presence of C and/or T; and at position 6911 is presence of T and/or C.

In one embodiment of the invention preferably the method for diagnosis described herein is one in which the polymorphism in the P2X₇ protein as defined by the position in SEQ ID NO: 4. is any one of the following: val76ala, his155tyr, val245gly, arg270his, arg276his, ala348thr, thr357ser, pro430arg, ala433val, gln460arg, ser490gly and glu496ala.

The method for diagnosis is preferably one in which the sequence is determined by a method selected from amplification refractory mutation system, restriction fragment length polymorphism and primer extension.

The status of the individual may be determined by reference to allelic variation at any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more positions.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| ELISA | Enzyme Linked ImmunoSorbent Assay |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

Table 1—Mutation Detection Techniques

General: DNA sequencing, Sequencing by hybridisation

Scanning: PTT*, SSCP, DGGE, TGGE, Cleavase, Heteroduplex analysis, CMC, Enzymatic mismatch cleavage *Note: not useful for detection of promoter polymorphisms.

Hybridisation Based

Solid phase hybridisation: Dot blots, MASDA, Reverse dot blots, Oligonucleotide arrays (DNA Chips).

Solution phase hybridisation: Taqman™—U.S. Pat. Nos. 5,210,015 & 5,487,972 (Hoffmann-La Roche), Molecular Beacons—Tyagi et al (1996), Nature Biotechnology, 14, 303; WO 95/13399 (Public Health Inst., New York)

Extension Based: ARMS™, ALEX™—European Patent No. EP 332435 B1 (Zeneca Limited), COPS—Gibbs et al (1989), Nucleic Acids Research, 17, 2347.

Incorporation Based: Mini-sequencing, APEX

Restriction Enzyme Based: RFLP, Restriction site generating PCR

Ligation Based: OLA

Other: Invader assay

Table 2—Signal Generation or Detection Systems

Fluorescence: FRET, Fluorescence quenching, Fluorescence polarisation—United Kingdom Patent No. 2228998 (Zeneca Limited)

Other: Chemiluminescence, Electrochemiluminescence, Raman, Radioactivity, Colorimetric, Hybridisation protection assay, Mass spectrometry

Table 3—Further Amplification Methods

SSR, NASBA, LCR, SDA, b-DNA

Table 4—Protein variation detection methods

Immunoassay

Immumunohistology

Peptide sequencing

Preferred mutation detection techniques include ARMS™, ALEX™, COPS, Taqman, Molecular Beacons, RFLP, and restriction site based PCR and FRET techniques. Immunoassay techniques are known in the art e.g. A Practical Guide to ELISA by D M Kemeny, Pergamon Press 1991; Principles and Practice of Imnmunoassay, 2 ^{nd} edition, C P Price & D J Newman, 1997, published by Stockton Press in USA & Canada and by Macmillan Reference in the United Kingdom. Histological techniques are described in Theory and Practice of Histological Techniques by J D Bancroft & A Stevens, 4^{th} Edition, Churchill Livingstone,1996. Protein sequencing is described in Laboratory Techniques in Biochemistry and Molecular Biology, Volume 9, Sequencing of Proteins and Peptides, G Allen, 2^{nd} revised edition, Elsevier, 1989. Particularly preferred methods include ARMS™ and RFLP based methods. ARMS ™ is an especially preferred method.

In a further aspect, the diagnostic methods of the invention are used to assess the pharmacogenetics of a drug acting at P2X₇.

Assays, for example reporter-based assays, may be devised to detect whether one or more of the above polymorphisms affect transcription levels and/or message stability.

Individuals who carry particular allelic variants of the P2X₇ gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The diagnostic methods of the invention may be useful both to predict the clinical response to such agents and to determine therapeutic dose.

In a further aspect, the diagnostic methods of the invention, are used to assess the predisposition and/or susceptibility of an individual to diseases mediated by P2X₇. This may be particularly relevant in the development of hyperlipoproteinemia and cardiovascular disease and the present invention may be used to recognise individuals who are particularly at risk from developing these conditions.

In a further aspect, the diagnostic methods of the invention are used in the development of new drug therapies which selectively target one or more allelic variants of the P2X₇ gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes.

According to another aspect of the present invention there is provided a human P2X₇ gene or its complementary strand comprising a variant allelic polymorphism at one or more of positions defined herein or a fragment thereof of at least 20 bases comprising at least one novel polymorphism.

Fragments are at least 17 bases, more preferably at least 20 bases, more preferably at least 30 bases.

According to another aspect of the present invention there is provided a polynucleotide comprising at least 20 bases of the human P2X₇ gene and comprising a polymorphism selected from any one of the following:

| Region | Polymorphism |
|---|---|
| | SEQ ID NO:1 |
| 5′ UTR | 936 C→A |
| | 1012 T→C |
| | 1147 A→G |
| | 1343 G→A |
| | 1476 A→G |
| | SEQ ID NO:2 |
| exon 2 | 253 T→C |
| exon 5 | 488 G→A |
| | 489 C→T |
| exon 7 | 760 T→G |
| exon 8 | 835 G→A |
| | 853 G→A |
| exon 11 | 1068 G→A |
| | 1096 C→G |
| exon 12 | 1315 C→G |
| exon 13 | 1324 C→T |
| | 1405 A→G |
| | 1448 C→T |
| | 1494 A→G |
| | 1513 A→C |
| | 1628 G→T |
| | 1772 G→A |
| | SEQ ID NO:3 |
| intron E | 4780 C→T |
| | 4845 C→T |
| | 4849 A→C |
| intron F | 5021 T→C |
| | 5554 (GTTT) n=3, 4 |
| | 5579 G→C |
| | 5535 A→T |
| intron G | 5845 C→T |
| | 6911 T→C |

According to another aspect of the present invention there is provided a polynucleotide comprising at least 20 bases of the human P2X₇ gene and comprising an allelic variant selected from any one of the following:

| Region | Variant |
|---|---|
| | SEQ ID NO:1 |
| 5′UTR | 936 A |
| | 1012 C |
| | 1147 G |
| | 1343 A |
| | 1476 G |
| | SEQ ID NO:2 |
| exon 2 | 253 C |
| exon 5 | 488 A |
| | 489 T |
| exon 7 | 760 G |
| exon 8 | 835 A |
| | 853 A |
| exon 11 | 1068 A |
| | 1096 G |
| exon 12 | 1315 G |
| exon 13 | 1324 T |
| | 1405 G |
| | 1448 T |
| | 1494 G |
| | 1513 C |
| | 1628 T |
| | 1772 A |
| | SEQ ID NO:3 |
| intron E | 4780 T |
| | 4845 T |
| | 4849 C |
| intron F | 5021 C |
| | 5554 (GTTT)ₙ, n=4 |
| | 5579 C |
| | 5535 T |
| intron G | 5845 T |
| | 6911 C |

According to another aspect of the present invention there is provided a human P2X₇ gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The invention further provides a nucleotide primer which can detect a polymorthism of the invention.

According to another aspect of the present invention there is provided an allele specific primer capable of detecting P2X₇ gene polymorphism, preferably at one more of the positions as defined herein.

An allele specific primer is used, generally together with a constant primer, in an amplifiction reaction such as a PCR reaction, which provides the discrimination between alleles through selective amplification of one allele at a particular sequence position e.g. as used for ARMS™ assay. The allele specific premier is preferably 17-50 nucleotides, more preferably about 17-35 nucleotides, more preferably about 17-30 nucleotides.

An allele specific primer preferably corresponds exacly with the allele to be detected but derivaties thereof are also contemplated wherein about 6-8of the nucleotides at the 3′ terminus correspond with the allele to be detected and wherein up to 10, such as up 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a P2X₇ gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a P2X₇ gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the polymorphisms of this invention may be used as genetic markers in linkage studies. This particularly applies to the polymorphisms of relatively high frequency. The P2X₇ gene is on chromosome 12q24 (Buell et al, Receptors and Channels, 1998, 5,347-354). Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2⁴ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWF:Ag levels: identification of three novel single nucleotide polymorphisms in the vWF gene promoter. Blood (1999) 93:4277-83, Keightley A M, Lam Y M, Brady J N, Cameron C L, Lillicrap D).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis.

According to another aspect of the present invention there is provided a method of treating a human in need of treatment with a drug acting at P2X₇ in which the method comprises:

i) diagnosis of a polymorphism in P2X₇ in the human, which diagnosis preferably comprises determining the sequence at one or more of the following positions:

positions 936, 1012, 1147, 1343 and 1476 in the 5′ UTR region of the P2X₇ gene as defined by the position in SEQ ID NO: 1;

positions 253, 488, 489, 760, 835, 853, 1068, 1096, 1315, 1324, 1405, 1448, 1494, 1513, 1628 and 1772 in the coding region of the P2X₇ gene as defined by the position in SEQ ID NO: 2; and

positions 4780, 4845, 4849, 5021, 5554, 5579, 5535, 5845 and 6911 in the intron region of the P2X₇ gene as defined by the position in SEQ ID NO: 3; and

positions 76, 155, 245, 270, 276, 348, 357, 430, 433, 460, 490 and 496 in the P2X₇ polypeptide as defined by the position in SEQ ID NO: 4;

and determining the status of the human by reference to polymorphism in P2X₇; and

ii) administering an effective amount of the drug.

Preferably determination of the status of the human is clinically useful. Examples of clinical usefulness include deciding which drug or drugs to administer and/or in deciding on the effective amount of the drug or drugs. The term “drug acting at P2X7” means that drug binding with P2X7 in humans is an important part of a drug exerting its pharmceutical effect in man. Compounds which are known to be antagonists of the P2X7 receptor are described in published PCT application Nos. WO 99/29660, WO 99/29661, WO 99/29686, WO 00/61569, WO 00/71529, WO 01/42194, WO 01/44170, WO 01/44213 and WO 01/46200.

According to another aspect of the present invention there is provided use of a drug acting at P2X₇ in preparation of a medicament for treating a disease in a human diagnosed as having a polymorphism therein, preferably at one or more of the positions defined herein.

According to another aspect of the present invention there is provided a pharmaceutical pack comprising P2X₇ drug and instructions for administration of the drug to humans diagnostically tested for a polymorphism therein, preferably at one or more of the positions defined herein.

According to another aspect of the present invention there is provided an allelic variant of human P2X₇ polypeptide comprising at least one of the following:

a alanine at position 76 of SEQ ID NO 4;

a tyrosine at position 155 of SEQ ID NO 4;

a glycine at position 245 of SEQ ID NO 4;

a histidine at position 270 of SEQ ID NO 4;

a histidine at position 276 of SEQ ID NO 4;

a threonine at position 348 of SEQ ID NO 4;

a serine at position 357 of SEQ ID NO 4;

a arginine at position 430 of SEQ ID NO 4;

a valine at position 433 of SEQ ID NO 4;

a arginine at position 460 of SEQ ID NO 4;

a glycine at position 490 of SEQ ID NO 4; and

a glutamic acid at position 496 of SEQ ID NO 4;

or a fragment thereof comprising at least 10 amino acids provided that the fragment comprises at least one allelic variant.

Fragments of polypeptide are at least 10 amino acids, more preferably at least 15 amino acids, more preferably at least 20 amino acids.

According to another aspect of the present invention there is provided an antibody specific for an allelic variant of human P2X₇ polypeptide as described herein.

Antibodies can be prepared using any suitable method. For example, purified polypeptide may be utilized to prepare specific antibodies. The term “antibodies” is meant to include polycional antibodies, monoclonal antibodies, and the various types of antibody constructs such as for example F(ab′)₂, Fab and single chain Fv. Antibodies are defined to be specifically binding if they bind the allelic variant of P2X₇ with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmnuassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a diagnostic kit rising an antibody of the invention.

According to another aspect of the present invention there is provided a polynucleotide comprising any one of the following twenty six P2X₇ haplotypes:

| | | | | | | | | |
|---|---|---|---|---|---|---|---|---|
| 1012 | 489 | 5579 | 835 | 853 | 1068 | 1096 | 1405 | 1513 |
| SEQ ID | SEQ ID | SEQ ID | SEQ ID | SEQ ID | SEQ ID | SEQ ID | SEQ ID | SEQ ID |
| 1 | 2 | 3 | 2 | 2 | 2 | 2 | 2 | 2 |

| | | | | | | | | | |
|---|---|---|---|---|---|---|---|---|---|
| 1 | T | T | C | G | G | A | G | A | A |
| 2 | C | C | G | G | G | G | C | A | A |
| 3 | C | C | C | A | G | G | C | A | C |
| 4 | C | T | G | G | G | A | C | G | A |
| 5 | C | C | G | G | G | A | G | A | A |
| 6 | C | C | C | A | G | G | C | A | A |
| 7 | T | T | G | G | G | A | C | G | A |
| 8 | C | T | C | G | G | G | C | A | A |
| 9 | C | C | C | G | G | A | C | A | A |
| 10 | C | T | G | G | G | G | C | A | C |
| 11 | T | C | G | G | G | A | C | A | A |
| 12 | C | T | C | G | G | G | C | A | C |
| 13 | T | C | C | G | G | A | C | A | A |
| 14 | T | C | C | G | G | G | C | A | C |
| 15 | C | T | C | G | G | A | C | A | A |
| 16 | T | T | C | G | G | A | C | G | A |
| 17 | C | C | G | G | G | A | C | G | A |
| 18 | T | C | G | A | A | G | C | A | A |
| 19 | C | C | C | G | G | G | G | A | A |
| 20 | T | C | C | G | G | G | G | A | A |
| 21 | C | T | C | A | G | G | C | A | A |
| 22 | C | C | C | G | G | G | C | A | C |
| 23 | C | T | G | G | A | A | G | G | A |
| 24 | T | T | G | G | G | A | G | G | A |
| 25 | C | T | C | G | G | G | G | A | A |
| 26 | C | C | C | G | G | G | C | A | A |

According to another aspect of the present invention there is provided a human P2X₇ polypeptide comprising one of the following eighteen combinations of alleleic variant determined amino acids based on positions identified in SEQ ID NO: 4:

| | 155 | 270 | 276 | 348 | 357 | 460 | 496 |
|---|---|---|---|---|---|---|---|
| 1 | Y | R | R | T | S | Q | E |
| 2 | Y | R | R | T | T | R | E |
| 3 | Y | R | R | T | T | Q | E |
| 4 | Y | R | R | T | S | R | E |
| 5 | Y | R | R | A | T | Q | A |
| 6 | Y | R | R | A | T | Q | E |
| 7 | Y | R | R | A | S | Q | E |
| 8 | Y | R | H | T | S | R | E |
| 9 | Y | H | R | A | T | Q | E |
| 10 | H | R | R | T | T | Q | E |
| 11 | H | R | R | T | T | R | E |
| 12 | H | R | R | A | T | Q | A |
| 13 | H | R | R | A | S | Q | E |
| 14 | H | R | R | A | T | Q | E |
| 15 | H | R | R | T | S | Q | E |
| 16 | H | H | R | A | T | Q | A |
| 17 | H | H | R | A | T | Q | E |
| 18 | H | H | H | A | T | Q | E |

According to another aspect of the present invention there is provided a polynucleotide codes any human P2X₇ polypeptide combination of allelic variants defined herein.

The invention will now be illustrated but not limited by reference to the following Examples. All temperatures are in degrees Celsius.

In the Examples below, unless otherwise stated, the following methodology and have been applied.

AMPLITAQ™, available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989) or “Current Protocols in Molecular Biology”, Volumes 1-3, Edited by F M Asubel, R Brent & R E Kingston, published by John Wiley, 1998.

Electropherograms were obtained in a standard manner: data was collected by ABI377 data collection software and the wave form generated by ABI Prism sequencing analysis (2.1.2).

EXAMPLE 1

Identification of Polymorphisms

1. Methods

DNA Preparation

DNA was prepared from frozen blood samples collected in EDTA following protocol I (Molecular Cloning: A Laboratory Manual, p392, Sambrook, Fritsch and Maniatis, 2^{nd} Edition, Cold Spring Harbor Press, 1989) with the following modifications. The thawed blood was diluted in an equal volume of standard saline citrate instead of phosphate buffered saline to remove lysed red blood cells. Samples were extracted with phenol, then phenol/chloroform and then chloroform rather than with three phenol extractions. The DNA was dissolved in deionised water.

Template Preparation

Templates were prepared by PCR using the oligonucleotide primers and annealing temperatures set out below. The extension temperature was 72° and denaturation temperature 94°. Generally 50 ng of genomic DNA was used in each reaction and subjected to 35 cycles of PCR. Where described below, the primary fragment was diluted 1/100 and two microliters were used as template for amplification of secondary fragments. PCR was performed in two stages (primary fragment then secondary fragment) to ensure specific amplification of the desired target sequence.

Polymorphisms in P2X₇

| | | | | protein | |
|---|---|---|---|---|---|
| Region | Size | Polymorphism | | change | frequency |
| 5′ UTR | | 936 | C→A | | 3/56 |
| | | 1012 | T→C | | 42/56 |
| | | 1147 | A→G | | 3/56 |
| | | | | | 2/52 |
| | | 1343 | G→A | | 35/52 |
| | | 1476 | A→G | | |
| exon 1 | 146bp | | | | |
| intron A | 21.7kb | | | | |
| exon 2 | 168bp | 253 | T→C | val76ala | 2/54 |
| intron B | 1.1kb | | | | |
| exon 3 | 68bp | | | | |
| intron C | 4.7kb | | | | |
| exon 4 | 73bp | | | | |
| intron D | 1.5kb | | | | |
| exon 5 | 95bp | 488 | G→A | silent | 2/54 |
| | | 489 | C→T | his155tyr | 17/38 |
| intron E | 2.8kb | 4780 | C→T | | 39/52 |
| | | 4845 | C→T | | 39/52 |
| | | 4849 | A→C | | 28/36 |
| exon 6 | 80bp | | | | |
| intron F | 617bp | 5021 | T→C | | 1/34 |
| | | 5554 | (GTTT) n=3, 4 | | n=3, |
| | | | | | 14/40 |
| | | 5579 | G→C | | 26/40 |
| | | 5535 | A→T | | 1/44 |
| exon 7 | 129bp | 760 | T→G | val245gly | 1/40 |
| intron G | 1.3kb | 5845 | C→T | | 2/40 |
| | | 6911 | T→C | | 33/50 |
| exon 8 | 136bp | 835 | G→A | arg270his | 16/52 |
| | | 853 | G→A | arg276his | 1/54 |
| intron H | | | | | |
| exon 9 | 91bp | | | | |
| intron I | 1.7kb | | | | |
| exon 10 | 64bp | | | | |
| intron J | 84bp | | | | |
| exon 11 | 149bp | 1068 | G→A | ala348thr | 18/62 |
| | | 1096 | C→G | thr357ser | 5/66 |
| intron K | | | | | |
| exon 12 | 101bp | 1315 | C→G | pro430arg, | 4/66 |
| | | | | splice site | |
| intron L | 3.8kb | | | | |
| exon 13 | 497bp | 1324 | C→T | ala433val | 1/54 |
| | | 1405 | A→G | gln460arg | 3/54 |
| | | 1448 | C→T | silent | 2/54 |
| | | 1494 | A→G | ser490gly | 2/54 |
| | | 1513 | A→C | glu496ala | 8/54 |
| | | | | silent | 2/52 |
| | | 1628 | G→T | silent | 24/54 |
| | | 1772 | G→A | | |

Positions in the 5′ UTR refer to SEQ ID NO: 1.

Positions in exons refer to SEQ ID NO: 2.

Positions in introns refer to SEQ ID NO: 3.

Positions in protein refer to SEQ ID NO: 4.

Evidence for effects of some polymorphisms on transaction are as follows. C at position 1012 SEQ ID No 1 disrupts the TCAAT motif from an enhancer binding sequence reported in intron 1 of EGFR. A at position 1147 SEQ ID No 1 disrupts the reverse sequence of the TCCTGC motif which is also an enchaner binding sequence from intron 1 EGFR. (Maekawa T., Imamoto F., Merino G. T., Pastran I., Ishii S. Cooperative Function of Two Separate Enhancers of RT the Human Epidermal Growth Factor Receptor Proto-oncogene J. Biol. Chem. 264:5488-5494 (1989)).

### EXAMPLE 2

Haplotype analysis

a) The following allele frequencies were determined in Swedish population.

| SEQ ID NO | Position | Frequency |
|---|---|---|
| 1 | 1012 | 46/60 |
| 2 | 489 | 27/60 |
| 3 | 5579 | 39/60 |
| 2 | 835 | 16/58 |
| 2 | 853 | 3/60 |
| 2 | 1068 | 24/58 |
| 2 | 1096 | 6/58 |
| 2 | 1045 | 11/60 |
| 2 | 1513 | 10/60 |

b) Hapolyte data.

Analysis of 15 Swedish families with at least one asthmatic child using primer extension (SNapShot™, Perkin Elmer) genotyping and GeneHunter® analysis demonstrated the following haplotypes:

| | | | | | | | | | | Fre- |
|---|---|---|---|---|---|---|---|---|---|---|
| | | | | | | | | | | quency |
| | 1012 | 489 | 5579 | 835 | 853 | 1068 | 1096 | 1405 | 1513 | n/58 |
| 1 | T | T | C | G | G | A | G | A | A | 1 |
| 2 | C | C | G | G | G | G | C | A | A | 3 |
| 3 | C | C | C | A | G | G | C | A | C | 1 |
| 4 | C | T | G | G | G | A | C | G | A | 5 |
| 5 | C | C | G | G | G | A | G | A | A | 1 |
| 6 | C | C | C | A | G | G | C | A | A | 8 |
| 7 | T | T | G | G | G | A | C | G | A | 1 |
| 8 | C | T | C | G | G | G | C | A | A | 3 |
| 9 | C | C | C | G | G | A | C | A | A | 3 |
| 10 | C | T | G | G | G | G | C | A | C | 2 |
| 11 | T | C | G | G | G | A | C | A | A | 2 |
| 12 | C | T | C | G | G | G | C | A | C | 3 |
| 13 | T | C | C | G | G | A | C | A | A | 4 |
| 14 | T | C | C | G | G | G | C | A | C | 1 |
| 15 | C | T | C | G | G | A | C | A | A | 2 |
| 16 | T | T | C | G | G | A | C | G | A | 1 |
| 17 | C | C | G | G | G | A | C | G | A | 2 |
| 18 | T | C | G | A | A | G | C | A | A | 2 |
| 19 | C | C | C | G | G | G | G | A | A | 1 |
| 20 | T | C | C | G | G | G | G | A | A | 1 |
| 21 | C | T | C | A | G | G | C | A | A | 4 |
| 22 | C | C | C | G | G | G | C | A | C | 3 |
| 23 | C | T | G | G | A | A | G | G | A | 1 |
| 24 | T | T | G | G | G | A | G | G | A | 1 |
| 25 | C | T | C | G | G | G | G | A | A | 1 |
| 26 | C | C | C | G | G | G | C | A | A | 1 |

This results in the following proteins:

| position | | | | | | | | |
|---|---|---|---|---|---|---|---|---|
| SEQ ID | | | | | | | | Frequency |
| NO 4 | 155 | 270 | 276 | 348 | 357 | 460 | 496 | N/58 |
| amino | Y | R | R | T | S | Q | E | 1 |
| acid | | | | | | | | |
| | Y | R | R | T | T | R | E | 7 |
| | Y | R | R | T | T | Q | E | 2 |
| | Y | R | R | T | S | R | E | 1 |
| | Y | R | R | A | T | Q | A | 5 |
| | Y | R | R | A | T | Q | E | 3 |
| | Y | R | R | A | S | Q | E | 1 |
| | Y | R | H | T | S | R | E | 1 |
| | Y | H | R | A | T | Q | E | 4 |
| | H | R | R | T | T | Q | E | 9 |
| | H | R | R | T | T | R | E | 2 |
| | H | R | R | A | T | Q | A | 4 |
| | H | R | R | A | S | Q | E | 3 |
| | H | R | R | A | T | Q | E | 3 |
| | H | R | R | T | S | Q | E | 1 |
| | H | H | R | A | T | Q | A | 1 |
| | H | H | R | A | T | Q | E | 8 |
| | H | H | H | A | T | Q | E | 2 |

c) Analysis

Ben J. Gu, Weiyi Zhang, Rebecca A. Worthington, Ronald Sluyter, Phuong Dao-Ung, Steven Petrou, Julian A. Barden, and James S. Wiley, J. Biol. Chem. (2001) 276: 11135-11142 reported that Ala at 496 (C at 1513) leads to loss of function in P2X7. Only one polymorphism was reported since they only analysed the final exon for SNPs

1. A method for the diagnosis of a polymorphism in P2X₇ in a human, which method comprises determining the sequence of the human at one or more of the following positions:
positions 936, 1012, 1147, 1343 and 1476 in the 5′ UTR region of the P2X₇ gene as defined by the position in SEQ ID NO: 1;
positions 253, 488, 489, 760, 835, 853, 1068, 1096, 1315, 1324, 1405, 1448, 1494, 1513, 1628 and 1772 in the coding region of the P2X₇ gene as defined by the position in SEQ ID NO: 2; and
positions 4780, 4845, 4849, 5021, 5554, 5579, 5535, 5845 and 6911 in the intron region of the P2X₇ gene as defined by the position in SEQ ID NO: 3;
positions 76 , 155, 245, 270, 276, 348, 357, 430, 433, 460, 490 and 496 in the P2X₇ polypeptide as defined by the position in SEQ ID NO: 4; and determining the status of the human by reference to polymorphism in P2X₇.

2. Use of a diagnostic method as defined in claim 1 to assess the pharmacogenetics of a drug acting at P2X₇.

3. A polynucleotide comprising at least 20 bases of the human P2X₇ gene and comprising an allelic variant selected from any one of the following:

| Region | Variant |
|---|---|
| | SEQ ID NO:1 |
| 5′ UTR | 936 A |
| | 1012 C |
| | 1147 G |
| | 1343 A |
| | 1476 G |
| | SEQ ID NO:2 |
| exon 2 | 253 C |
| exon 5 | 488 A |
| | 489 T |
| exon 7 | 760 G |
| exon 8 | 835 A |
| | 853 A |
| exon 11 | 1068 A |
| | 1096 G |
| exon 12 | 1315 G |
| exon 13 | 1324 T |
| | 1405 G |
| | 1448 T |
| | 1494 G |
| | 1513 C |
| | 1628 T |
| | 1772 A |
| | SEQ ID NO:3 |
| intron E | 4780 T |
| | 4845 T |
| | 4849 C |
| intron F | 5021 C |
| | 5554 (GTTT)ₙ, n=4 |
| | 5579 C |
| | 5535 T |
| intron G | 5845 T |
| | 6911 C |

4. A nucleotide primer which can detect a polymorphism as defined in claim 1.

5. An allele specific primer capable of detecting a P2X₇ gene polymorphism as defined in claim 1.

6. An allele-specific oligonucleotide probe capable of detecting a P2X₇ gene polymorphism as defined in claim 1.

7. Use of a P2X₇ gene polymorphism as defined in claim 1 as a genetic marker in a linkage study.

8. A method of treating a human in need of treatment with a drug acting at P2X₇ in which the method comprises:
i) diagnosis of a polymorphism in P2X₇ in the human, which diagnosis preferably comprises determining the sequence at one or more of the following positions:
positions 936, 1012, 1147, 1343 and 1476 in the 5′UTR region of the P2X₇ gene as defined by the position in SEQ ID NO: 1;
positions 253, 488, 489, 760, 835, 853, 1068, 1096, 1315, 1324, 1405, 1448, 1494, 1513, 1628 and 1772 in the coding region of the P2X₇ gene as defined by the position in SEQ ID NO: 2; and positions 4780, 4845, 4849, 5021, 5554, 5579, 5535, 5845 and 6911 in the intron region of the P2X₇ gene as defined by the position in SEQ ID NO: 3; and positions 76, 155, 245, 270, 276, 348, 357, 430, 433, 460, 490 and 496 in the P2X₇ polypeptide as defined by the position in SEQ ID NO: 4;
and determining the status of the human by reference to polymorphism in P2X₇ ; and
ii) administering an effective amount of the drug.

9. An allelic variant of human P2X₇polypeptide comprising at least one of the following:
a alanine at position 76 of SEQ ID NO 4;
a tyrosine at position 155 of SEQ ID NO 4;
a glycine at position 245 of SEQ ID NO 4;
a histidine at position 270 of SEQ ID NO 4;
a histidine at position 276 of SEQ ID NO 4;
a threonine at position 348 of SEQ ID NO 4;
a serine at position 357 of SEQ ID NO 4;
a arginine at position 430 of SEQ ID NO 4;
a valine at position 433 of SEQ ID NO 4;
a arginine at position 460 of SEQ ID NO 4;
a glycine at position 490 of SEQ ID NO 4; and
a glutamic acid at position 496 of SEQ ID NO 4;
or a fragment thereof comprising at least 10 amino acids provided that the fragment comprises least one allelic variant.

10. An antibody specific for an allelic variant of human P2X₇ polypeptide as defined in claim 9.

11. A polynucleotide comprising any one of the following twenty six P2X₇ haplotypes:

| | | | | | | | | |
|---|---|---|---|---|---|---|---|---|
| 1012 | 489 | 5579 | 835 | 853 | 1068 | 1096 | 1405 | 1513 |
| SEQ ID | SEQ ID | SEQ ID | SEQ ID | SEQ ID | SEQ ID | SEQ ID | SEQ ID | SEQ ID |
| 1 | 2 | 3 | 2 | 2 | 2 | 2 | 2 | 2 |
| | | | | | | | | | |
|---|---|---|---|---|---|---|---|---|---|
| 1 | T | T | C | G | G | A | G | A | A |
| 2 | C | C | G | G | G | G | C | A | A |
| 3 | C | C | C | A | G | G | C | A | C |
| 4 | C | T | G | G | G | A | C | G | A |
| 5 | C | C | G | G | G | A | G | A | A |
| 6 | C | C | C | A | G | G | C | A | A |
| 7 | T | T | G | G | G | A | C | G | A |
| 8 | C | T | C | G | G | G | C | A | A |
| 9 | C | C | C | G | G | A | C | A | A |
| 10 | C | T | G | G | G | G | C | A | C |
| 11 | T | C | G | G | G | A | C | A | A |
| 12 | C | T | C | G | G | G | C | A | C |
| 13 | T | C | C | G | G | A | C | A | A |
| 14 | T | C | C | G | G | G | C | A | C |
| 15 | C | T | C | G | G | A | C | A | A |
| 16 | T | T | C | G | G | A | C | G | A |
| 17 | C | C | G | G | G | A | C | G | A |
| 18 | T | C | G | A | A | G | C | A | A |
| 19 | C | C | C | G | G | G | G | A | A |
| 20 | T | C | C | G | G | G | G | A | A |
| 21 | C | T | C | A | G | G | C | A | A |
| 22 | C | C | C | G | G | G | C | A | C |
| 23 | C | T | G | G | A | A | G | G | A |
| 24 | T | T | G | G | G | A | G | G | A |
| 25 | C | T | C | G | G | G | G | A | A |
| 26 | C | C | C | G | G | G | C | A | A |

12. A human P2X₇ polypeptide comprising one of the following eighteen combinations of alleleic variant determined amino acids based on positions identified in SEQ ID NO: 4:

| | 155 | 270 | 276 | 348 | 357 | 460 | 496 |
|---|---|---|---|---|---|---|---|
| 1 | Y | R | R | T | S | Q | E |
| 2 | Y | R | R | T | T | R | E |
| 3 | Y | R | R | T | T | Q | E |
| 4 | Y | R | R | T | S | R | E |
| 5 | Y | R | R | A | T | Q | A |
| 6 | Y | R | R | A | T | Q | E |
| 7 | Y | R | R | A | S | Q | E |
| 8 | Y | R | H | T | S | R | E |
| 9 | Y | H | R | A | T | Q | E |
| 10 | H | R | R | T | T | Q | E |
| 11 | H | R | R | T | T | R | E |
| 12 | H | R | R | A | T | Q | A |
| 13 | H | R | R | A | S | Q | E |
| 14 | H | R | R | A | T | Q | E |
| 15 | H | R | R | T | S | Q | E |
| 16 | H | H | R | A | T | Q | A |
| 17 | H | H | R | A | T | Q | E |
| 18 | H | H | H | A | T | Q | E |

13. A polynucleotide which encodes any human P2X₇ polypeptide as defined in claim 12.
